(19) United States
(12) Patent Application Publication
(10) Pub. No.: US 2023/0346557 A1
(43) Pub. Date: Nov. 2, 2023

(54) STENT DELIVERY SYSTEM FOR USE IN AORTA

(71) Applicant: SHANGHAI FLOWDYNAMICS MEDICAL TECHNOLOGY CO., LTD., Shanghai (CN)

(72) Inventors: Jian DING, Shanghai (CN); Chenying FAN, Shanghai (CN)

(73) Assignee: SHANGHAI FLOWDYNAMICS MEDICAL TECHNOLOGY CO., LTD., Shanghai (CN)

(21) Appl. No.: 18/003,925

(22) PCT Filed: Nov. 6, 2020
(86) PCT No.: PCT/CN2020/127161

§ 371 Date: Dec. 30, 2022

(30) Foreign Application Priority Data
Jul. 6, 2020 (CN) 202010642752.3

(51) Int. Cl. A61F 2/24

(52) U.S. Cl. CPC A61F 2/2466; A61F 2240/001

(57) ABSTRACT

A stent delivery system for use in an aorta, including: a delivery catheter comprising an outer catheter, an inner catheter, and a push rod that are arranged coaxially and sequentially from outside to inside, and a stent. The stent is a dense-mesh stent. The delivery catheter is configured to cause the outer catheter, the inner catheter, and the push rod to all be able to move relative to one another in the axial direction, and two ends of the stent being removably bound to the proximal end relative to the heart of the push rod and the proximal end relative to the heart of the inner catheter. When the stent delivery system of the disclosure is used in treating aortic aneurysms and/or aortic dissections, the force required for initial release of the stent may be reduced, and local axial compression of the partially released stent may be controlled.

### TECHNICAL FIELD

The disclosure relates to a stent delivery system and a method for placing a stent by the stent delivery system, and in particular to a stent delivery system for use in the treatment of aortic lesions, such as aortic aneurysms or aortic dissections.

### BACKGROUND

A wall of an arterial blood vessel is composed of intima, tunica media and adventitia which are in tight contact with one other. When an inner wall of the arterial blood vessel is partially damaged, the tunica media of the wall of the arterial blood vessel is gradually peeled off under a strong impact of arterial blood flow, so that blood enters between the tunica media and the adventitia of the wall of the blood vessel to form two lumens, i.e., a true lumen and a false lumen. Aortic dissection is most commonly seen. The aortic dissection causes the arterial wall to become thin and weak, with a risk of rupture at any time. Once the dissection is ruptured, the patient will die in several minutes.

The aortic dissection is divided into two types, i.e., type A and type B (i.e., Stanford typing) according to a tearing site and an extension range of the intima. Type A refers to a lesion involving an ascending aorta, and a peeling site of the arterial wall starts from the ascending aorta, and may also occur at a proximal end of an aortic arch or of a descending aorta but involves the ascending aorta. Type B means that a peeling site of the arterial wall occurs at the descending aorta without exceeding a proximal end of an opening of a left subclavian artery.

The aortic aneurysm is also a disease of abnormal expansion of an aorta. Rupture of the aortic aneurysm is also fatal for the patient.

Therefore, it is very necessary to early diagnose and timely treat the aortic dissection and the aortic aneurysm.

A conventional solution is an EVAR intraluminal intervention method, i.e., a covered stent implantation. This solution has advantages of small trauma, fast recovery and low mortality rate. However, a site where a stent is placed often involves an aortic arch and/or an abdominal aorta, three branch artery blood vessels at a convex side of the aortic arch, and main branch artery blood vessels such as a left renal artery, a right renal artery, a coeliac trunk artery, a superior mesenteric artery, or the like at the abdominal aorta cannot be obstructed during the whole surgery and after the surgery. Conventional practice is to drill a hole at a site of the covered stent corresponding to an important branch artery. On one hand, difficulty of surgery is increased, and the surgery needs to be implemented by a surgeon with rich experience; and on the other hand, once positioning of the stent is not accurate during placement, or the stent is displaced during release, an important branch blood vessel may be obstructed to induce serious consequences. Furthermore, the stent is modified before surgery, and the manufacturer thereof may refuse to provide a warranty service for this reason.

Recently, there is also proposed a solution in which total aortic intraluminal intervention is performed by using a dense mesh stent (i.e., TEVAR intervention method). Unlike the EVAR intraluminal intervention method, this solution does not use a mechanism of mechanically obstructing a false lumen, and the dense mesh stent does not significantly hinder passage of blood flow. Instead, by forming obstruction of blood flow at an inner wall of a lesion blood vessel, hemodynamics in the false lumen are changed, the pressure in the false lumen is reduced, and intraluminal thrombus is promoted, so as to achieve the purpose of treatment. Compared with the EVAR intraluminal intervention method, since this solution uses the dense mesh stent, it not only has advantages of small trauma, fast recovery and low mortality rate, but also the dense mesh stent does not significantly obstruct delivery of blood flow to a branch artery, thereby greatly reducing difficulty of surgery. Therefore, a doctor with ordinary experience may implement this solution.

However, a treatment effect of this type of stent is not ideal enough. Since a tear of the inner wall of the blood vessel is not completely obstructed, an ideal intraluminal thrombus cannot be always formed. Furthermore, existing dense mesh stents are still difficult to be applied to all lesion types of aortic dissection and aortic aneurysm, especially difficult to be applied to type A aortic lesion.

Type A aortic dissection involves the ascending aorta, and an inner diameter of the blood vessel at this site significantly increases due to lesion, usually up to 38 mm to 55 mm. After such dense mesh stent with a large diameter is radially compressed to a delivery configuration with a very small diameter, a frictional force between the stent and a wall of a delivery catheter is significantly increased usually due to a large self-expanding force of the stent, which leads to the need for a greater thrust to release the stent. The increase of a release force of the stent makes it difficult to control the accuracy of a minimally invasive surgical implemented by remote manipulation. This increased frictional force especially makes it difficult to release the stent at a site of the ascending aorta. Since an aortic blood vessel turns almost 180° at the aortic arch, a stent delivery system entering the ascending aorta from a distal end of the aorta also turns almost 180°. When the stent is initially released, a direction of pushing force applied by a side of an operator also changes by approximately 180° when the pushing force reaches a most front end of the delivery system in the ascending aorta, which makes the operator more uncontrollable in releasing the stent in the ascending aorta, thereby increasing difficulty and risk of surgery.

### SUMMARY

In view of this, a main purpose of the disclosure is to provide a stent delivery system capable of solving or improving at least one of the above problems in the related art. Specifically, a purpose of the disclosure is to provide a stent delivery system intended to treat the aortic dissection or aortic aneurysm, especially type A aortic lesion. The stent delivery system has a reduced stent initial release force, and partially axial compression and/or stretching is performed on a partially released stent, to provide different radial support forces and/or fluid permeability at different segments of different treatment sites.

To this end, a first aspect of the disclosure provides a stent delivery system, including a delivery catheter and a stent.

The delivery catheter includes an outer catheter, an inner catheter and a push rod which are coaxially arranged sequentially from exterior to interior of the delivery catheter.

The outer catheter is provided with a proximal end and a distal end, and is provided with a first hollow cavity penetrating through the outer catheter.

The inner catheter extends in the first hollow cavity at the distal end of the outer catheter, and is provided with a second hollow cavity penetrating through the inner catheter.

The push rod extends through the first hollow cavity of the outer catheter and the second hollow cavity of the inner catheter, and extends out of a distal end of the inner catheter.

The stent is a dense mesh stent, and is releasably maintained in the first hollow cavity between the outer catheter and the push rod at the proximal end of the outer catheter in a delivery configuration.

The delivery catheter is configured so that the outer catheter, the inner catheter and the push rod are axially movable relative to one another, and one of two ends of the stent is removably constrained to a proximal end of the push rod, and another of the two ends of the stent is removably constrained to a proximal end of the inner catheter.

In the stent delivery system of the disclosure, one of two ends (i.e., cardiac proximal and distal ends) of the stent is removably constrained to the proximal end of the push rod, and another of the two ends of the stent is removably constrained to the proximal end of the inner catheter. According to a method for placing a stent as will be described in detail below, when the stent is released, the outer catheter is maintained stationary and the push rod is pushed in a direction toward the heart, so that the push rod may drive the stent and the inner catheter to move in the direction toward the heart, and correspondingly, the outer catheter moves in a direction away from the heart, thus the stent is released from the proximal end of the stent. Since the proximal end of the stent is constrained to the proximal end of the push rod, a frictional resistance between the stent and the outer catheter is reduced since the end of the stent is constrained during initial release. Reduction of the frictional resistance is more conducive to release of the stent, especially for release of the stent in the ascending aorta. Since the delivery catheter turns nearly 180° after passing through the aortic arch, a push direction of the push rod at a side of the operator is almost opposite to a movement direction of the push rod at a side of the ascending aorta. Therefore, the smaller a friction force between the stent and the outer catheter, the smaller a force used by the operator to push the push rod, and the easier it is for the operator to control a release process, thereby facilitating a stable release of the stent.

Furthermore, since the proximal end of the stent is constrained to the proximal end of the push rod, the stent is not fully expanded and is in a semi-release state during initial release. At this time, the diameter of the stent does not reach a diameter in a full release state thereof, and does not abut against a wall of the blood vessel, so that it is easy to adjust position of the stent. Furthermore, for example, a partially released stent may be retracted into the delivery catheter, and released again after its position is adjusted.

When at least a part of the stent is released, one end of the stent is maintained stationary, and another end of the stent is moved toward the one end (for example, the inner catheter and the outer catheter are maintained stationary, the push rod is pulled back), so that the released part of the stent may be compressed. In this way, said part has significantly increased metal coverage and radial support force, thereby achieving functions of obstructing a tear at a tearing site of an inner wall of the blood vessel and supporting a true lumen of the blood vessel. On the other hand, one end of the stent is maintained stationary, and another end of the stent is moved in a direction away from the one end (for example, the push rod is maintained stationary, the inner catheter and the outer catheter are pulled back), so that the released part of the stent may be stretched, thereby maintaining a smooth blood flow at a site such as a site having a branch artery.

One of the two ends of the stent may be temporarily fixed to the push rod by a stopper, and another of the two ends of the stent may be temporarily fixed to the inner catheter by a stopper. After the stent is properly released, the stoppers are removed from the stent finally, so that the two ends of the stent are released to complete placement of the stent.

Components configured to constrain two ends of the stent are not specifically limited in the disclosure, and may be any existing component which may be removed finally to release the two ends of the stent.

A dense mesh stent placed at a treatment site by using the stent delivery system of the disclosure has different degrees of compression in a length direction thereof, so that the stent may have different metal coverage and radial support forces, while meeting requirements of obstructing the tear and not obstructing delivery of blood flow to a branch blood vessel.

The stent delivery system of the disclosure may have a conventional outer diameter, thereby facilitating passing through a femoral artery to place the stent a site of the aorta needing to be treated. According to the disclosure, even as to a stent to be released in the ascending aorta, the stent may also be assembled in the delivery catheter by axially extending and radially compressing the stent to a suitable diameter.

According to an embodiment, the outer catheter of the stent delivery system of the disclosure may have an outer diameter of about 5 mm to about 10 mm, preferably an outer diameter of about 5 mm to about 7 mm.

According to an embodiment, the push rod is hollow for the passage of guide wires.

The stent delivery system of the disclosure is suitable for delivery of a dense mesh bare stent.

According to a preferred embodiment, the stent may be a stent which is at least partially compressible and extendable along an axial direction of the stent in a natural release state (i.e., without subject to axial compression and without extension), and has a radial support force of greater than or equal to 100 N and a metal coverage of at least 30%.

When the above stent is used, the delivery system of the disclosure may partially axially compress the stent. When the stent is released in the blood vessel, a required performance of liquid impermeability is obtained substantially in a partial segment of a non-branch blood vessel of the stent by partially axial compression, to obstruct the tear and achieve a radial support force of expanding the true lumen. In particular, at a tearing site of intima of the blood vessel, more particularly in the ascending aorta, a partially enhanced radial support force and a significantly reduced fluid (blood) permeability may be obtained by axially compressing the partially released stent. On the other hand, at a site where a branch artery is present, meshes of the stent may become sparse through partially axial extension if necessary, thereby facilitating delivery of blood to the branch artery, and facilitating placement of a branch stent through the meshes if necessary. Furthermore, due to the above characteristics of the stent, density of the woven wires may be significantly reduced after the stent is axially extended, and then the stent is radially compressed, so that the stent is easily compressed into a delivery configuration with a suitable diameter, and may be conveniently assembled into the delivery system of the disclosure. This is conducive for the stent delivery system of the disclosure to have the suitable diameter, to easily implement placement of the stent through the femoral artery.

According to an embodiment, the stent may be formed by weaving at least two kinds of first wires and second wires with different diameters, here each of the first wires has a diameter of 20 μm to 150 μm and each of the second wires has a diameter of 150 μm to 800 μm.

The stent is formed by weaving at least two kinds of wires with different diameters. On one hand, this enables a large-sized stent used in the aorta to have a moderate radial support force and metal coverage, and to be radially compressed to a suitable delivery dimension. On the other hand, the thicker second wires form a basic skeleton and perform a function of providing a basic radial support force, and the thinner first wires may effectively fill gaps between the second wires and perform functions of assisting support and maintaining the shape of the stent. In particular, after the stent is axially compressed, fluid permeability of the compressed part may be significantly reduced due to presence of the first wires, to form effective obstruction on the tearing site of the inner wall of the blood vessel. In addition, after the thicker second wires are axially compressed, density of the second wires per unit length is increased, so that a significantly enhanced radial support force may be formed, thereby perform a function of expanding the true lumen of the blood vessel and helping to reduce the false lumen.

According to a specific embodiment, the stent may have a radial support force of 100 N to 600 N and a metal coverage of 30% to 90% in the natural release state.

According to an embodiment, the stent may have a radial support force of greater than or equal to 400 N, preferably a radial support force of 400 N to 1000 N, and a metal coverage of 80% to 100%, preferably a metal coverage of 80% to 95%, in a release and axial maximum compression state.

In the range of the radial support force in the natural release state as defined above, the stent of the disclosure may be easily assembled into a delivery system with a suitable outer diameter (for example, 5 mm to 10 mm) even though the stent has a maximum diameter (such as 38 mm to 60 mm) suitable for the ascending aorta. Furthermore, after the stent is maximum compressed axially, the stent has a radial support force in the above range, so that it may effectively support a narrowed true lumen of the blood vessel. In particular, a radial support force in the preferred range is particularly conducive to a lesion site which has been formed for a certain period of time, where a torn intima of the blood vessel becomes hard and requires a greater force to be expanded.

Therefore, in an actual application, when the stent of the disclosure is released in the blood vessel (especially the aorta), the stent has different compression ratios in a length direction thereof, and radial support forces of the stent at different sites may vary in a range of greater than or equal to 100 N, preferably a range of 100 N to 1000 N.

According to an embodiment, the stent may have a metal coverage of 30% to 90% in the natural release state.

According to an embodiment, the stent has a substantially complete liquid permeability in the natural release state, that is, the stent does not form obstruction of blood flow from the aorta to the branch artery substantially or completely. According to the embodiment, the stent has a radial support force of 200 N to 600 N and a metal coverage of 30% to 60% in the natural release state. The stent having a radial support force and a metal coverage in above specified ranges has a relatively low radial support force and a relatively low metal coverage in the natural release state, but has a relatively large compressible ratio. The stent may still form necessary radial support force and metal coverage meeting the above provisions after it is compressed, to effectively obstruct a tear of the inner wall of the blood vessel.

According to another embodiment, the stent has a radial support force of 100 N to 600 N and a metal coverage of 60% to 90%, preferably a metal coverage of 70% to 90%, in the natural release state. The stent having a radial support force and a metal coverage in above specified ranges has a relatively high radial support force and a relatively high metal coverage in the natural release state, but has a relatively large extendable ratio, and the stent may still be radially compressed to a suitable dimension after it is extended, to be assembled into a suitable delivery system. The stent of the embodiment has a relatively large radial support force and a relatively large metal coverage without compression after it is partially released, thereby facilitating immediate support and expansion of the true lumen of the blood vessel, and obstructing the tear to a certain extent. Further, the radial support force may be further enhanced and the metal coverage may be increased by axially compressing a partial segment of the stent, to form necessary radial support force and metal coverage meeting the above provisions, so as to obstruct the tear of the inner wall of the blood vessel more effectively.

According to the embodiment, the stent has a certain liquid permeability in the natural release state, that is, the stent obstructs blood flow from the aorta to the branch artery to some extent. In a specific embodiment, since the stent according to the disclosure is axially extendable, a suitable blood permeability may be obtained by properly stretching a partial segment corresponding to the branch vessel when the stent is placed by using the delivery system of the disclosure. To this end, a corresponding part of the stent is configured to have an increased diameter relative to an adjacent part thereof, so that the diameter of this area after it is stretched is suitable for an inner diameter of a lumen of the blood vessel having a branch blood vessel. In another specific embodiment, the stent may be provided with at least one sparse mesh area formed only of the second wires, and the at least one sparse mesh area is arranged at a site of a corresponding treatment site having a branch artery after the stent is released. According to one solution, the treatment site includes an aortic arch, and the sparse mesh area includes a first sparse mesh area. The first sparse mesh area corresponds to a greater curvature of the aortic arch, especially corresponds to a branch artery on the aortic arch: a brachiocephalic trunk artery, a common left neck artery, and a left subclavian artery. Specifically, a length of the first sparse mesh area is greater than a length of the branch arterial area on the aortic arch, such as 6 cm to 8 cm, and the first sparse mesh area corresponds to a circumferential angle of about 120° to about 180°. According to another solution, the treatment site includes an abdominal aorta, and the sparse mesh area includes a second sparse mesh area. The second sparse mesh area corresponds to a branch artery part of the abdominal aorta, especially corresponds to a branch artery on the abdominal aorta: a left renal artery, a right renal artery, a coeliac trunk artery, and a superior mesenteric artery. Specifically, a length of the second sparse mesh area is greater than a length of the branch arterial distribution area on the abdominal aorta, such as 2 cm to 4 cm, and the second sparse mesh area corresponds to a circumferential angle of about 180°. In different solutions, the stent may be provided with the first sparse mesh area and/or the second sparse mesh area.

In the embodiment, any mesh of each sparse mesh area is expandable, allowing the branch stent to be placed through the expanded mesh.

The stent according to the embodiment may be woven according to requirements of the metal coverage of other parts of the stent, and after weaving is completed, the first wires are removed in an area where the sparse mesh area is predetermined to be formed, to form the sparse mesh area.

According to an embodiment, the stent may be used in an aorta including an abdominal aorta site, and the stent may be internally provided with two common iliac artery stent fixing parts configured to fix left and right common iliac artery stents.

According to a specific embodiment, the common iliac artery stent fixing parts may be arranged inside the stent and correspond to the abdominal aorta close to a bifurcation of left and right common iliac arteries, and the two common iliac artery stent fixing parts may be configured as two annuluses tangent to each other and may be integrally formed with an inner wall of the stent.

Unlike fixing parts forming two cylindrical branches at a lower part of a stent for the abdominal aorta, the two fixing parts of the stent of the disclosure are arranged inside the stent, so that the exterior of the stent is still maintained to be cylindrical, which is more conducive to expanding the blood vessel, without poor support near the common iliac arteries. Furthermore, the common iliac artery fixing parts are integrally formed with the stent, to fix left and right iliac artery stents more stably.

According to an embodiment, each of the first wires for the dense mesh stent may have a diameter of 50 μm to 150 μm; and each of the second wires may have a diameter of 150 μm to 600 μm.

According to an embodiment, the stent is formed by weaving three kinds of wires with different diameters, here the second wires may include at least one first thick wire and at least one second thick wire with different diameters. According to a specific embodiment, the at least one first thick wire may have a diameter of 150 μm to 300 μm, and the at least one second thick wire may have a diameter of 300 μm to 600 μm.

According to another embodiment, the stent is formed by weaving three kinds of wires with different diameters, here the first wires may include at least one first thin wire and at least one second thin wire with different diameters. According to a specific embodiment, the at least one first thin wire may have a diameter of 20 μm to 100 μm, and the at least one second thin wire may have a diameter of 100 μm to 150 μm.

According to still another embodiment, the stent is formed by weaving four kinds of wires with different diameters, i.e., the first and second thin wires as well as the first and second thick wires.

The number of wires for weaving the stent may be 48 to 202, preferably 64 to 196. Here the number of the second wires may be 4 or more, such as 4 to 32, preferably 4 to 30, and the remaining wires are the first wires.

According to an embodiment, the second wires include 6 to 24 first thick wires and at least four second thick wires, and the remaining wires are the first wires.

In case of too much number of thick wires (i.e., the second wires), the stent cannot be effectively compressed into an ideal delivery state, while in case of too small number of thick wires, the stent cannot provide an expected radial support force even after compression, and an expected structure and morphology of the stent cannot be maintained in a release state. In particular, as to the stent of the disclosure, the number of the second wires preferably is less than or equal to 24.

According to an embodiment, the first wires may include wires with two diameters. In the embodiment, the number of wires for weaving the stent may be 48 to 202. Here the number of the second wires may be 4 to 32, preferably 4 to 30, and the remaining wires are the first wires. The first wires may include 32 to 166 first thin wires and 32 to 166 second thin wires, provided that a sum of the number of the first thin wires and the number of the second thin wires is less than or equal to 198.

According to an embodiment, in order to obtain a greater metal coverage and/or a greater radial support force, the stent may be provided with at least two layers of woven meshes. According to an embodiment, the stent is provided with two to four layers of woven meshes. When the stent is provided with multiple layers of woven meshes, the stent as a whole has the radial support force and the metal coverage as specified above.

A method for weaving the stent of the disclosure is not specifically limited. A conventional overlapping and weaving may be used (that is, there is no constraint at an intersection between the wires), as long as it is possible to facilitate axial compression and stretching of the stent.

It should be understood that according to a range of the radial support force and a range of the metal coverage required for a specific treatment type, those skilled in the art may select a suitable woven material and determine a reasonable number of layers under specific device conditions according to descriptions herein, further select a suitable diameter, number, or the like of the wire, and determine a suitable weaving solution (such as weaving density) to obtain a stent having the required range of the radial support force and the required range of the metal coverage. For example, a suitable weaving solution may be designed by a dedicated software.

According to a further embodiment, an end (especially the proximal end where the stent has a maximum radial support force) of the stent may be formed in a return weaving manner. As for another end of the stent, if there is a burr which cannot be woven again in a return weaving manner, the burr may be located on an inner side of the stent by selecting a suitable arrangement of the layers; or if there are two layers of burrs, the burr at one layer of the two layers is woven in a return weaving manner by a short segment, and the other burr is wrapped in said segment which is woven in a return weaving manner, or when multiple stents are used in cooperation, burrs at a single layer or two layers may be overlapped with each other and placed in another dense mesh stent. The stent according to the embodiment has a smooth end, thereby avoiding mechanical damage to the inner wall of the blood vessel by an exposed end (burr) of the wire.

The stent may have the same diameter as a whole, or the stent may have a variable diameter, and the stent may have the diameter ranging from 20 mm to 60 mm, preferably ranging from 20 mm to 55 mm.

According to an embodiment, the stent of the disclosure in a release state is provided with a first segment arranged at the proximal end of the stent. The first segment may have a diameter of 38 mm to 60 mm to be suitable for the ascending aorta. The first segment has a length of 8 cm to 11 cm. The stent may be further provided with a second segment adjacent to the first segment. The second segment may have a diameter of 25 mm to 35 mm to be suitable for a part of the artery from the aortic arch to the abdominal aorta. Following the first segment, the second segment extends to the distal end of the stent. The second segment may have a length of 25 cm to 45 cm, preferably 25 cm to 31 cm.

According to another embodiment, the stent may have a diameter of 20 mm to 30 mm to be suitable for a part of the abdominal aorta. In the embodiment, the stent may have a length of 15 cm to 25 cm, preferably 15 cm to 20 cm.

The stent of the disclosure is self-expandable or capsular expandable. Materials of the first wires and materials of the second wires for weaving the stent may be different from each other, however, preferably the same. Usually, the materials of the wires may be metal, such as stainless steel, shape memory alloy (e.g., nitinol), cobalt-chromium alloy, tungsten, or tantalum.

According to a second aspect of the disclosure, there is also provided a method for placing a stent by using the stent delivery system of the disclosure, which includes the following operations.

The stent delivery system according to the disclosure is guided to a treatment site.

At least a part of the stent is released.

One end of the released part of the stent is maintained stationary, and the stent delivery system is manipulated, so that another end of the released part moves along an axial direction of the stent to compress/extend the released part.

After the stent is completely released, constraints on two ends of the stent are removed.

According to the method for placing the stent, the position of the stent is adjusted after the stent is released for a certain length, and the push rod may be pulled back in a direction away from the heart when the position of the inner catheter and the position of the outer catheter are stationary for example (or, the inner catheter and the outer catheter are pushed in a direction close to the heart when position of the push rod is stationary), so that the semi-released stent is driven to be compressed axially. Such axial compression is caused due to the fact that in the stent delivery system of the disclosure, two ends of the stent are constrained, so that when one of the two ends is stationary and another end of the two ends is moved, the released part of the stent may be axially compressed/stretched. With the stent delivery system of the disclosure, the released segment of the stent may be compressed to have its maximum metal coverage and maximum radial support force, so that it tightly abuts against a wall of the blood vessel at a corresponding site (especially a tearing site of the intima) under the action of its radial support force, to complete release of this segment.

Similarly, other parts of the stent may be selected to be completely released directly as desired, or may be released while compressed/stretched in segments. After the release substantially completes finally, constraints on the two ends of the stent are removed, and the delivery catheter together with the push rod is withdrawn from the lumen of the blood vessel.

The method for placing the stent of the disclosure further includes the following operations. It is detected whether a release position of the stent is correct after at least a part of the stent according to the disclosure is released.

When it is found that the release position is incorrect, the stent delivery system is adjusted so that the released part is located at a correct position, or the released part is retracted into the delivery catheter, the stent delivery system is adjusted to the correct position, and the stent is released again.

Since in the stent delivery system of the disclosure, the two ends of the stent are temporarily constrained, the stent may be compressed or stretched in segments during release of the stent. In particular, as to the tearing site of the inner wall of the blood vessel, effective obstruction and a required radial support force may be formed at this local area by compression operations to achieve a best therapeutic effect. Furthermore, other parts or specific parts of the stent do not hinder blood flow, and does not create a risk caused by interruption or deficiency of blood supply to the branch artery.

Optionally, according to an embodiment, in the branch arterial area, a larger mesh may also be formed at this local area by stretching operations, to facilitate placing the branch stent in the branch blood vessel. According to an embodiment, in the method, the operation that at least a part of the stent is released includes an operation that the sent is released from a proximal end of the stent.

According to an embodiment, in the method, the proximal end portion of the stent is compressed. Since a tear of the aortic dissection usually occurs at an end of the dissection close to the heart, the proximal end portion of the stent is usually required to be compressed, to obstruct the tear.

According to the method of the disclosure, the delivery catheter is withdrawn from the treatment site after placement of the stent is completed.

According to an embodiment, the method further includes the following operations. The stent delivery system is introduced from a femoral artery of the subject, and the delivery catheter is withdrawn from the femoral artery of the subject after the stent is placed.

According to an embodiment, the method further includes the following operations. The stent delivery system is guided to the treatment site by guide wires.

According to an embodiment, the subject is a mammal, preferably a pig or a person, more preferably a person, most preferably a person with yellow race.

According to an embodiment, the treatment site is any site from the ascending aorta to the abdominal aorta. According to a specific embodiment, the treatment site includes a tearing site of the intima of the blood vessel. According to a more specific embodiment, the treatment site further includes a branch vessel.

The stent delivery system of the disclosure is configured to treat the aortic dissection or aortic aneurysm of the subject.

### BRIEF DESCRIPTION OF THE DRAWINGS

FIG. 1 is a schematic view of a stent delivery system according to the disclosure.

FIG. 2 is a schematic view of a stent delivery system of the disclosure in a partial release state.

FIG. 3 is a schematic view illustrating partially axial compression and stretching of a stent according to the disclosure.

FIG. 4 is a schematic view illustrating a process of placing a stent in an aorta with a tear by using a stent delivery system of the disclosure.

FIG. 5 is a schematic view illustrating placing a stent in a type A aortic dissection lesion by using a stent delivery system of the disclosure.

FIG. 6 is a schematic view and partially enlarged view of a stent applicable to an embodiment of a stent delivery system of the disclosure.

FIG. 7 is a schematic view of a stent applicable to another embodiment of a stent delivery system of the disclosure.

FIG. 8 is a schematic view of a stent applicable to yet another embodiment of a stent delivery system of the disclosure.

FIG. 9 is a schematic partially enlarged view of a stent applicable to still another embodiment of a stent delivery system of the disclosure.

FIG. 10 is a schematic cross-section view of a woven layer of a stent with multiple layers according to the disclosure.

### DETAILED DESCRIPTION

Technical solutions in embodiments of the disclosure will be clearly and completely described below in combination with the embodiments of the disclosure and the drawings. It is apparent that the described embodiments are only part of the embodiments of the disclosure, rather than all the embodiments, and the technical solutions recited in the embodiments of the disclosure may be implemented in any combination without conflict. All other embodiments obtained by those of ordinary skill in the art based on the embodiments of the disclosure without paying any creative work belong to the scope of protection of the disclosure.

Throughout the description, terms used here should be understood as meanings as usually used in the art, unless specifically stated otherwise. Therefore, unless defined otherwise, all technical and scientific terms used here have same meanings as usually understood by those skilled in the art to which the disclosure belongs. When there is a contradiction, meanings of the description are preferred.

Like reference numerals in the drawings refer to like components. Shapes and dimensions of components in schematic drawings are for illustration only and cannot be considered to reflect actual shapes, dimensions and absolute positions.

It should be noted that in the disclosure, terms “including”, “include”, or any other variants thereof are intended to cover a non-exclusive inclusion, so that a method or device including a series of elements not only includes elements which are explicitly recited, but also includes other elements which are not explicitly listed, or further includes elements inherent to implementation of the method or device.

It should be noted that terms “first \ second” involved in the disclosure are only intended to distinguish similar objects, and do not represent a specific sequence of the objects, and it may be understood that “first \ second” may exchange a specific sequence or order in an allowable situation. It should be understood that objects distinguished by “first \ second” may be interchanged in an appropriate situation, to enable embodiments of the disclosure described here to be implemented in an order other than those illustrated or described here.

In order to describe the disclosure more clearly, terms “proximal end” and “distal end” are customary terms in the field of intervention medical treatment. Here “distal end” represents an end away from the heart during surgical operation, and “proximal end” represents an end close to the heart during surgical operation.

The disclosure provides a stent delivery system. In the delivery system, a dense mesh stent is assembled in the system in a delivery configuration, and both ends of the stent are constrained. The constraints are removed only after other parts of the stent are released, so that the stent is completely released.

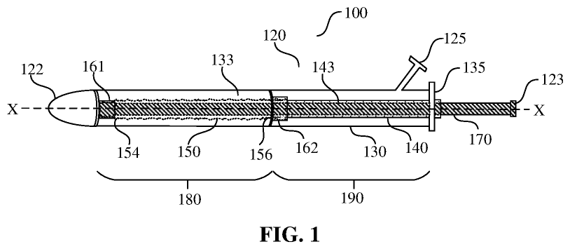

With reference to FIG. 1, a schematic view of a stent delivery system 100 according to the disclosure is shown. The stent delivery system 100 includes a delivery catheter 120 and a stent 150.

The delivery catheter 120 includes an outer catheter 130, an inner catheter 140 and a push rod 170 which are coaxially arranged sequentially from exterior to interior of the delivery catheter along a longitudinal axis X-X. The delivery catheter 120 is provided with a distal end 123 and a proximal end 122. The delivery catheter 120 is further provided with a hemostatic valve 125. The outer catheter 130 is provided with a proximal end 180 and a distal end 190, and a first hollow cavity 133 penetrates through the whole outer catheter 130. The inner catheter 140 is arranged coaxially with the outer catheter 130 along the longitudinal axis X-X in the first hollow cavity 133 at the distal end 190 of the outer catheter, and the inner catheter 140 is provided with a second hollow cavity 143. The push rod 170 extends through the first hollow cavity 133 of the outer catheter 130 and extends through the second hollow cavity 143 of the inner catheter 140, until extending beyond a port 135 of the distal end of the outer catheter. The push rod 170 may be provided with a third hollow cavity (not shown) for the passage of guide wires.

At the proximal end 180 of the outer catheter 130, the stent 150 is releasably maintained in the first hollow cavity 133 between the push rod 170 and the outer catheter 130 in a delivery configuration. A proximal end 154 of the stent 150 is constrained at a proximal end of the push rod 170 by a first constraint component 161. The first constraint component 161 may be a conventional stopper which may be removed from the stent 150 if necessary, to release the proximal end 154 of the stent 150. A distal end 156 of the stent 150 is constrained at a proximal end of the inner catheter 140 by a second constraint component 162. Similarly, the second constraint component 162 may be a conventional stopper which may be removed from the stent 150 if necessary, to release the distal end 156 of the stent 150.

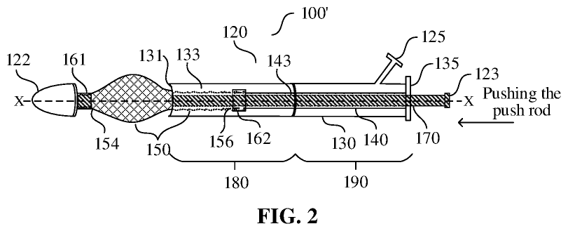

With further reference to FIG. 2, the stent delivery system shown in FIG. 1 in a semi-release state is shown. In FIG. 2, a proximal end 131 of the outer catheter 130 in the delivery system 100′ may be separated from the proximal end 122 of the delivery catheter 120, the outer catheter 130 may move relative to the inner catheter 140 and the push rod 170 by maintaining the outer catheter stationary and pushing the push rod 170 toward the proximal end (or by maintaining the inner catheter and the push rod stationary and pulling the outer catheter 130 toward the distal end).

In the system 100′ shown in FIG. 2, by pushing the push rod 170 toward the proximal end 122, the proximal end 122 of the delivery catheter 120 drives the push rod 170 fixedly connected to the proximal end 122, the stent 150 constrained together with an end of the push rod 170 and the inner catheter 140 constrained together with the stent 150 to move toward the proximal end relative to the outer catheter 130. In this way, the stent 150 is started to be released from the proximal end 154 thereof.

Since the proximal end 154 of the stent 150 is constrained during release, the diameter of the released segment does not reach the diameter thereof in a natural release state thereof, and the released segment is not in contact with a wall of the blood vessel. On one hand, it is conducive to adjusting the position of the stent, and on the other hand, the stent 150 may also be retracted into the outer catheter 130 again, and the stent 150 is released again after its position is adjusted. Furthermore, the proximal end 154 of the stent 150 is constrained, which is also conducive to reducing a friction force during initial release and conducive to a stable release. This is particularly important for surgery in which distal operations are performed in a narrow blood vessel.

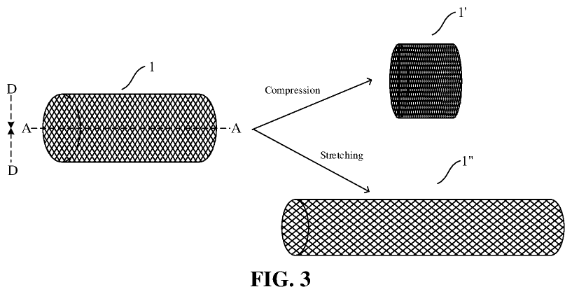

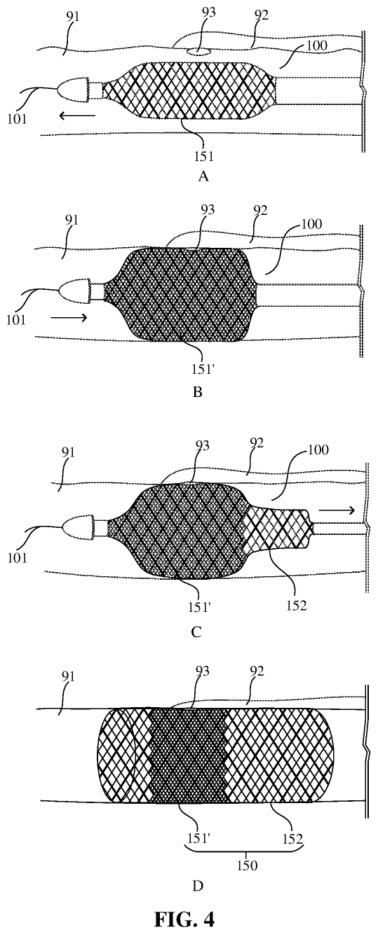

A method for releasing a stent in an aorta by using the stent delivery system of the disclosure is described in further detail in combination with FIG. 3 and FIG. 4.

FIG. 3 schematically shows cases in which the stent is compressed and stretched respectively. The stent in the stent delivery system of the disclosure is a dense mesh stent. In a further embodiment, the stent is at least partially compressible and extendable along an axial direction of the stent in a natural release state.

As shown in FIG. 3, in case that one end of the stent is fixed, the stent 1 may be axially compressed along a central axis A-A of the stent 1 into a compressed stent 1′, or the stent 1 may be axially stretched into a stretched stent 1″. The compressed stent 1′ has a significantly increased metal coverage, thereby obtaining a significantly reduced fluid permeability and a significantly increased radial support force, to be adapted to a tearing site of intima of the blood vessel. The stretched stent 1″ has a significantly reduced metal coverage, so that in some cases, the stent is more readily compressed radially, to obtain a suitable delivery configuration, which is conducive to assembling a stent with a larger diameter into the stent delivery system of the disclosure.

The expression “axial direction” of the stent as mentioned here refers to a direction along A-A as shown in FIG. 3, which is a direction of a central axis of a cylindrical shape of the stent. The expression “radial direction” of the stent as mentioned here refers to a direction along D-D as shown in FIG. 3, which is a diameter direction of a circle of the cylindrical shape of the stent. In general, “radially compressed” here refers to compression in a direction from the circumference to the center of the circle.

The expression “natural release state” of the stent as mentioned here refers to a state in which the stent is not axially compressed or stretched and is not radially compressed in a water bath at 37±2° C.

The expression “axial maximum compression state” of the stent as mentioned here refers to a state in which the stent is axially compressed until it is unable to be further compressed, when it is in the natural release state.

Further in combination with FIG. 4, a schematic view illustrating placing a stent at a tear of intima of an aorta by using the stent delivery system of the disclosure is shown. The part A of FIG. 4 shows a segment of aortic blood vessel 91 including a tear 93 of intima of the blood vessel, and tearing of the intima is induced by the tear 93 to form a false lumen 92. The stent delivery system 100 of the disclosure has been guided to the tear 93, and has released a segment 151 of the stent by pushing the push rod in a direction indicated by the arrow in case that the position of the outer catheter 130 remains unchanged. With further reference to the part B of FIG. 4, the position of the inner catheter and the position of the outer catheter of the delivery system 100 remain unchanged, and the push rod is pulled back in a direction indicated by the arrow in this figure, so that the released segment 151 of the stent is compressed back and finally abuts against a blood vessel area in which the tear 93 of the intima of the blood vessel is located, to form a compressed segment 151′. When the stent is designed, the diameter of the stent is usually designed to be slightly greater than the diameter of the blood vessel at a placement site. Therefore, the compressed segment 151′ tightly abuts against an inner wall of this segment of the blood vessel, obstructs the tear 93, and expands a true lumen of this segment of the blood vessel. The compressed segment 151′ is remained in this segment of the blood vessel with the compressed shape due to an inward contraction force caused by the wall of the blood vessel.

Next, as shown in the part C of FIG. 4, the remaining segment 152 of the stent is further released by pulling the outer catheter 130 toward the distal end in a direction indicated by the arrow in this figure (the inner catheter 140 and the push rod 170 remain stationary). Finally, after the stent is completely released, constraints of the stoppers on both ends of the stent are removed, so that the whole stent 150 is released to the treatment site, and the delivery catheter is withdrawn from the blood vessel (see the part D of FIG. 4). The stent 150 placed at a lesion site is provided with two segments, one of which is the compressed segment 151′, and the other of which is a natural release segment 152. The compressed segment 151′ performs functions of obstructing the tear 93 and forming relatively strong radial support on the blood vessel. The natural release segment 152 performs a function of supporting other parts of the blood vessel appropriately, and does not hinder flow of blood, in particular flow of blood toward a branch blood vessel.

According to the disclosure, after the segment 151 of the stent is released (i.e., a state shown in the part A of FIG. 4), the position of the stent may be confirmed, so that the tear 93 may be accurately obstructed by the compressed segment. If said position is not ideal enough, adjustment may be performed, and even the released segment 151 is retracted into the outer catheter. The stent is released again after the position of the delivery system is adjusted.

Similarly, the position of the stent may be confirmed after any segment of the stent is released, to achieve a best placement effect. Finally, the position of the stent is confirmed again before constraints on both ends of the stent are removed, since the stent may be retracted and released again when it is found that its placement position is not ideal at this time. After constraints on both ends of the stent are removed, the position of the stent cannot be adjusted.

Furthermore, other segments of the stent are compressed in a manner similar to that shown in the part B of FIG. 4. For example, when a front part of the stent is released and has abutted against the wall of the blood vessel, a segment of the stent continues to be released. Since a subsequently released end of the stent is constrained by the outer catheter, by remaining the push rod stationary and simultaneously moving the outer catheter and the inner catheter toward the proximal end, a segment, which is newly released but has not yet abutted against the wall of the blood vessel, of the stent is compressed and abuts against the wall of the blood vessel, to form a segment with a high metal coverage and a high radial support force.

The stent delivery system 100 of the disclosure is particularly advantageous for the treatment of type A aortic dissection.

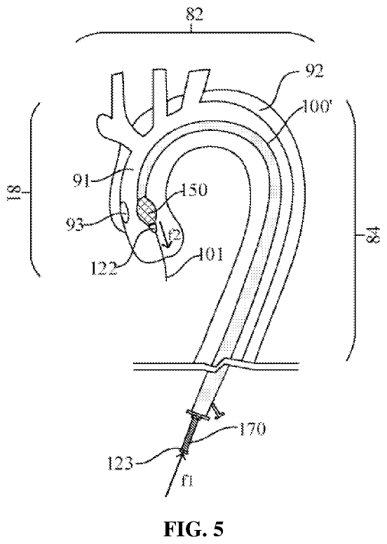

With reference to FIG. 5, a schematic view illustrating placing a stent 150 in a type A aortic dissection lesion by using the stent delivery system 100′ of the disclosure is shown, to explain advantages of the treatment of the stent delivery system of the disclosure on the type A aortic dissection. FIG. 5 shows that there is a tear 93 in an ascending aorta 81, thus a false lumen 92 is formed from the ascending aorta 81 through an aortic arch 83 to a descending aorta 84. The stent delivery system 100′ of the disclosure has been guided into a true lumen 91 of the aorta by a guide wire 101. When the stent 150 is started to be released, the operator pushes the push rod 170 in a direction toward the proximal end, where the thrust is represented by f1 and a direction of the thrust is indicated by an arrow directed upward along the descending aorta. The thrust f1 is transmitted to the proximal end 122 of the delivery catheter along the push rod 170. At this time, since the delivery system 100′ turns nearly 180° after passing through the aortic arch 83, a direction of a force f2 acting on the proximal end 122 of the delivery catheter is almost opposite to the direction of the thrust at the distal end 123 of the delivery catheter (as indicated by the arrow). This causes difficulty for controlling a magnitude of the thrust required during initial release of the stent. In the stent delivery system 100′ of the disclosure (see FIG. 1 and FIG. 2), the proximal end of the stent 150 is temporarily constrained at the proximal end of the push rod 170 by a component such as a stopper, thereby significantly reducing an outward expansion force of the stent 150 at the proximal end, and reducing the friction force between the stent 150 and an inner wall of the outer catheter 130. Therefore, the thrust required when the stent is initially released by the delivery system 100′ of the disclosure is significantly reduced, and the proximal end of the push rod 170 may be easily pushed out of the outer catheter 130.

The stent delivery system of the disclosure is particularly suitable to be assembled with a stent which is at least partially compressible and extendable along an axial direction of the stent in a natural release state. Advantageously, the stent has a radial support force of greater than or equal to 100 N and a metal coverage of 3090%, to provide a of delivery configuration with a suitable dimension, as well as suitable fluid permeability and radial support force after partial compression.

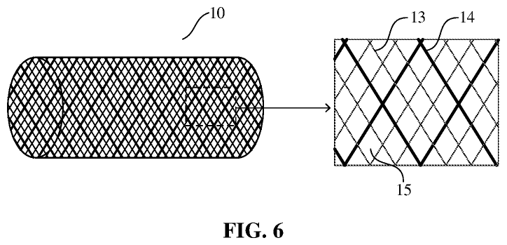

According to an embodiment, the stent is formed by weaving at least two kinds of first wires and second wires with different diameters, here each of the first wires has a diameter of 20 μm to 150 μm and each of the second wires has a diameter of 150 μm to 800 μm. FIG. 6 shows a schematic view and partially enlarged view of a stent 10 according to the embodiment. The stent 10 is formed by overlapping and weaving multiple first wires 13 and multiple second wires 14. The stent 10 shown in FIG. 6 is a single-layer mesh structure with meshes 15. The stent of the embodiment is more suitable for the stent delivery system of the disclosure. Since two kinds of wires with different diameters mainly achieve functions of reducing fluid permeability and enhancing radial support force respectively in a large-size stent for the aorta, the stent may be compressed to a suitable delivery configuration to be assembled into the stent delivery system for the aorta according to the disclosure, while advantages of the two aspects are obtained.

The stent may also be a structure with multiple layers, such as 2 to 4 layers. For example, the multiple layers may be formed in a return weaving manner.

The stent of the disclosure is suitable for any segment from the ascending aorta to the abdominal aorta or even the whole aorta. Therefore, the stent of the disclosure may have a large diameter ranging from about 55 mm to about 20 mm.

The stent 10 may be formed by weaving a total of 48 to 202 wires, preferably 64 to 196 wires. For example, by way of enumeration, a stent of the disclosure may be formed by weaving 48, 96, 128, 156, 196 wires. The number of the wires may be determined according to the diameter of the stent, the number of layers, materials of the used wires, or the like.

Material for the stent may be any material suitable for a peripheral vascular stent, as long as the material may provide a sufficient radial support force and have a certain fineness. Usually, metal wires such as stainless steel wires, nickel-titanium alloy wires, cobalt-chromium alloy wires, tungsten wires, tantalum wires, or the like are preferably used, and stainless steel wires are preferred.

There are at least four second wires 14 serving as thick wires, and usually the number of the second wires 14 is less than or equal to 30. The diameter of each second wire 14 is included between 150 μm and 800 μm, preferably between 150 μm and 600 μm. The diameter of each second wire is for example 150 μm, 200 μm, 250 μm, 300 μm, 350 μm, 400 μm, 450 μm, 500 μm, 550 μm, or 600 μm. The second wires 14 provide a basic support force and a complete structure for the stent 10. However, the number of the second wires cannot be too much. For example, even though wires with the diameter of only 300 μm are used, when about 32 wires are used, the stent is difficult to be compressed to a suitable delivery dimension and thus is unable to use, especially for a stent part with ultra-large diameter greater than about 40 mm.

In the stent 10, the remaining wires except the thick wires are first wires 13 serving as thin wires. Each first wire 13 of the disclosure may have a diameter of 20 μm to 150 μm preferably a diameter of 50 μm to 150 μm such as 50 μm 60 μm 70 μm 80 μm 90 μm 100 μm 110 μm 1120 μm 130 μm 140 μm and 150 μm. The first wires 13 perform functions of auxiliary supporting the stent 10 and filling gaps between the second wires 14. Furthermore, the first wires 13 also perform a function of maintaining the shape of the stent 10, since the number of the first wires is much greater than the number of the second wires 14. The inventors have found that although it seems to be feasible theoretically, in fact, when the overlapping and weaving manner of the disclosure is used, a large-diameter stent with a certain shape and a sufficient support force cannot be formed by second wires (i.e., thick wires) alone. Furthermore, even if diameters of the wires are the same, a large-diameter stent with a fixed wire-to-wire intersection formed by using for example a laser engraving technology has a much smaller support force, which cannot achieve requirements for aortic blood vessels according to the disclosure.

The expression “radial support force” of the stent as mentioned here refers to a force required to compress the stent along a diameter direction to have 85% of its original diameter, after it is fixed in a water bath at 37±2° C. The radial support force may be measured by a radial support force tester.

Along with the radial support force, the stent also requires a high liquid permeability to obtain an effect of effectively obstructing a tear of the intima of the blood vessel. This property may be represented by metal coverage. The stent 10 may have a metal coverage of 30% to 90% in the natural release state, and the stent 10 may have a metal coverage of 80% to 100%, preferably a metal coverage of 80% to 95%, in the axial maximum compression state.

The expression “metal coverage” of the stent as mentioned here refers to a metal coverage ratio per unit area, which is measured by electron microscope scanning. A sum of the metal coverage and a void ratio per unit area should be 100%.

According to an embodiment, the stent may have a radial support force of 200 N to 600 N and a metal coverage of 30% to 60% in the natural release state. In the embodiment, the stent has a relatively low weaving density, and the stent also has a relatively low radial support force in the natural release state. In the embodiment, the stent has a high axial compressible ratio, and the required radial support force and metal coverage may still be obtained after the stent is compressed. When the stent is placed, a relatively long segment of the stent is required to be released and then compressed, and this requires the blood vessel to have a longer straight segment. Therefore, the stent is not suitable for a site of the blood vessel such as the ascending aorta in which a turn is present.

According to another embodiment, the stent may have a radial support force of 100 N to 600 N and a metal coverage of 60% to 90%, preferably a metal coverage of 70% to 90%, in the natural release state. In the embodiment, the stent has a relatively high weaving density, and the stent also has a relatively high radial support force in the natural release state. In the embodiment, the stent has a low axial compressible ratio, and a certain radial support force and metal coverage may be obtained without compression after it is released. The stent is suitable to be placed in a site with a shorter straight segment in the blood vessel such as the ascending aorta.

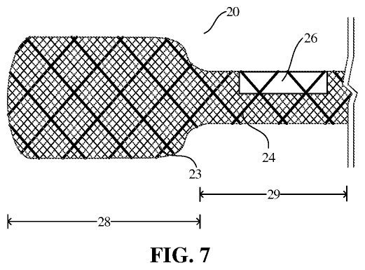

The stent of the embodiment has a relatively high metal coverage in the natural release state, and hinders blood flow to a certain extent. In order to maintain smooth blood flow at the branch artery, the stent of the embodiment has at least one sparse mesh area. With reference to FIG. 7, a stent 20 with a single-layer structure of the embodiment suitable to be placed at sites from the ascending aorta to the aortic arch is schematically shown. The stent 20 may be provided with multiple layers, such as 4 layers, to obtain a metal coverage in the above range. The stent 20 includes a segment 28 adapted to the ascending aorta and a segment 29 adapted to the aortic arch. The segment 28 adapted to the ascending aorta has a diameter ranging from about 38 mm to about 60 mm, and a diameter of the segment 29 adapted to the aortic arch is reduced to range from about 25 mm to about 35 mm. The segment 29 adapted to the aortic arch is provided with a sparse mesh area 26. Only the second wires (i.e., thick wires) 24 are arranged in the sparse mesh area 26. A part other than the sparse mesh area 26 is also provided with the first wires (i.e., thin wires) 23 in addition to the second wires. The sparse mesh area 26 is arranged at a site corresponding to a greater curvature of the aortic arch after the stent is released, and has an area larger than an opening of the branch vessel of the greater curvature in the artery. Typically, the sparse mesh area 26 may have a length of about 6 to 8 cm, and occupy about ⅓ arc length of the circumference of the segment 29 of the stent 20 with a central angle corresponding to about 120°.

According to another example, the stent (not shown) may be adapted to the whole arterial area from the ascending aorta to the abdominal aorta. After a segment of the ascending aorta, the stent is adapted to a segment which may extend from an area corresponding to the aortic arch to an area corresponding to the abdominal aorta, and the diameter of the stent may vary from about 25 mm˜about 35 mm to about 20 mm˜about 30 mm. The segment may include two sparse mesh areas formed of only the second wires, i.e., a first sparse mesh area corresponding to a branch artery (a brachiocephalic trunk artery, a common left neck artery, and a left subclavian artery) at the aortic arch, and a second sparse mesh area corresponding to a branch artery (a left renal artery, a right renal artery, a coeliac trunk artery, and a superior mesenteric artery) at the abdominal aorta. The first sparse mesh area may have a dimension in the embodiment as shown in FIG. 3. The second sparse mesh area may have a length of about 2 to 4 cm, and occupy about ½ arc length of the circumference of the segment with a central angle corresponding to about 180°.

The stent of the embodiment may be woven in a conventional manner, and then the first wires in a predetermined area are removed to form the sparse mesh area.

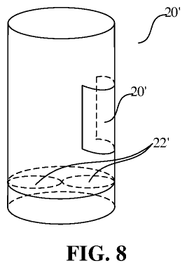

According to a further embodiment, at a part of the stent corresponding to the abdominal aorta which is the treatment site, the stent of the disclosure may be provided with two common iliac artery stent fixing parts configured to receive and fix left and right common iliac artery stents. With reference to FIG. 8, a stent of the embodiment is schematically shown. The stent 20′ shown in FIG. 8 is suitable for a segment of the abdominal aorta and is provided with a sparse mesh area 27′. According to other embodiments, the stent 20′ may not be provided with a sparse mesh area 27′. Common iliac artery stent fixing parts 22′ arranged as two adjacent annular channels are provided at interior of a lower part of the stent 20′. For the sake of clarity, each fixing part 22′ is shown as a plane in FIG. 4, and in fact, each fixing part 22′ has a certain thickness. In some examples, each common iliac artery stent fixing part 22′ may extend downward to a lower end of the stent 20′.

Each common iliac artery stent fixing part 22′ is also formed by weaving the same first and second wires as the stent 20′ (the first and second wires are not shown), and is integrally formed with the stent. Outer parts of the two annular channels are integrally woven with an inner wall of the stent 20′. The dimensions of inner diameters of the two annular channels are adapted to outer diameters of the left and right common iliac artery stents to be received and fixed, and are usually slightly less than the outer diameters of the left and right common iliac artery stents, so as to be able to fix the common iliac artery stents.

Of course, the embodiment in which the common iliac artery stent fixing parts are provided is also applicable to a stent without a sparse mesh area.

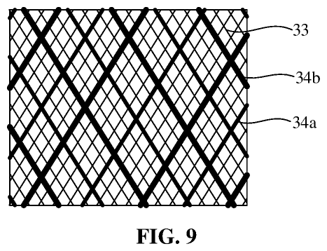

According to an embodiment, the stent of the disclosure may be formed by weaving three kinds of wires with different diameters. As shown in FIG. 9, a schematic partially enlarged view of a single layer of a stent of an example is shown. The stent is formed by weaving a kind of first wire 33 and two kinds of second wires 34a, 34b. Here the first wire 33 is a thin wire which may have a diameter of 50 μm to 150 μm; the second wires include a first thick wire 34a which may have a diameter of 150 μm to 300 μm and a second thick wire 34b which may have a diameter of 300 μm to 600 μm. Here the number of the first thick wire may be 6 to 12, the number of the second thick wire may be more than 4, but no more than 12 at most, and the remaining wires form the first wire. The embodiment may also have other variations. For example, the stent is formed by two kinds of first wires (for example, their diameters are in a range of 20 μm to 100 μm and a range of 100 μm to 150 μm, respectively) and a kind of second wire, or formed by two kinds of first wires and two kinds of second wires, but is not limited thereto.

A radial support force of the stent formed by three or more kinds of wires with different diameters is more uniform in the whole stent, and flexibility of the stent may also be enhanced.

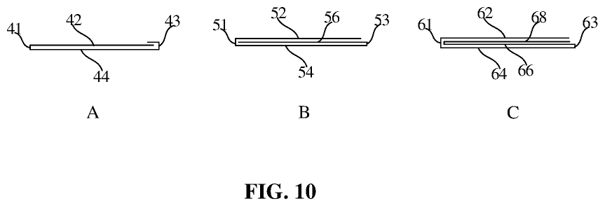

The stent may be provided with multiple layers, preferably two layers, three layers, or four layers. According to a preferred embodiment, the stent with multiple layers may be formed by way of weaving a single-layer woven mesh in a return weaving manner. As shown in the parts A to C of FIG. 10, schematic cross-section views of woven layers of stents with two to four layers are shown. The part A of FIG. 10 shows a structure with two layers, and in this figure, an upper layer 42 of the structure is located at a side of the stent close to the interior of the stent, and a lower layer 44 of the stent is located at a side of the stent close to exterior (i.e., a side in contact with the wall of the blood vessel) of the stent. A smooth port is formed at a proximal end 41 of the stent due to the return weaving. At a distal end 43 of the structure, the lower layer 44 facing toward the exterior of the stent is woven in a return weaving manner by a certain distance, to wrap, inside the lower layer 44, an opened edge (burr) of a distal end of the upper layer 42 facing toward the interior of the stent. Thus, a port with both smooth ends is formed. Similarly, in the part B of FIG. 10, an upper layer 52 is woven at a proximal end 51 in a return weaving manner to form a lower layer 54 and is further woven at a distal end 53 in a return weaving manner to form an intermediate layer 56. At the distal end 53, the lower layer 54 and the intermediate layer 56 are slightly longer than the upper layer 52, so that an opened edge of a distal end of the upper layer is located inside the stent. In this way, both ends of the stent are also smooth ports. The same principle is also applied to four layers in the part C of FIG. 10. At a distal end 63, two layers 62, 68 close to the interior of the stent are shorter than an edge formed by weaving two layers 64, 66 close to the exterior of the stent in a return weaving manner, while a proximal end 61 forms a lowermost layer 64 by weaving an uppermost layer 62 in a return weaving manner, to wrap two intermediate layers 68 and 66 therebetween. A variation may be possible that a distal end of the uppermost layer 62 or intermediate layer 68 is woven in a return weaving manner by a small segment, to wrap an opened edge of another layer. In this way, both ends of the stent are completely smooth ports.

Preferably, the stent of the disclosure is in the form of multiple layers, so that both ends (especially the proximal end) of the stent may form smooth ports, avoiding secondary damage to the blood vessel by an opened edge of the braid.

The stent of the disclosure is described in detail as above by way of examples. It should be understood by those skilled in the art that the above examples are intended to explain advantages of the stent of the disclosure, rather than limiting the scope of the disclosure, and features in an example may be applied to the stent of another example separately or in combination in an appropriate situation. Apparent variations and modifications made to the stent by those skilled in the art according to contents of the disclosure here fall within the scope of the disclosure, as long as they meet the concept of the disclosure.

Detailed solutions of the stent delivery system of the disclosure and the method for placing the stent into the blood vessel by using the system are explained by the above examples. Variations and modifications may be readily made by those skilled in the art based on the above contents, to be adapted to actual application requirements without departing from the spirit of the disclosure. These variations and modifications also fall within the scope of the disclosure.

1. A stent delivery system, wherein the stent delivery system is used in an aorta, the stent delivery system comprising:
a delivery catheter, wherein the delivery catheter comprises an outer catheter, an inner catheter and a push rod which are coaxially arranged sequentially from exterior to interior of the delivery catheter,
wherein the outer catheter is provided with a proximal end and a distal end, and is provided with a first hollow cavity penetrating through the outer catheter,
the inner catheter extends in the first hollow cavity at the distal end of the outer catheter, and is provided with a second hollow cavity penetrating through the inner catheter,
the push rod extends through the first hollow cavity of the outer catheter and the second hollow cavity of the inner catheter, and extends out of a distal end of the inner catheter; and
a stent, wherein the stent is a dense mesh stent, and releasably maintained in the first hollow cavity between the outer catheter and the push rod at the proximal end of the outer catheter in a delivery configuration,
wherein the delivery catheter is configured so that the outer catheter, the inner catheter and the push rod are axially movable relative to one another, and wherein one of two ends of the stent is removably constrained to a proximal end of the push rod, and another of the two ends of the stent is removably constrained to a proximal end of the inner catheter.

2. The stent delivery system of claim 1, wherein the outer catheter has an outer diameter of about 5 mm to about 10 mm, preferably an outer diameter of about 5 mm to about 7 mm.

3. The stent delivery system of claim 1, wherein one of the two ends of the stent is removably constrained to the proximal end of the push rod by a stopper, and another of the two ends of the stent is removably constrained to the proximal end of the inner catheter by a stopper.

4. The stent delivery system of claim 1, wherein the stent is a stent which is at least partially compressible and extendable along an axial direction of the stent in a natural release state, and has a radial support force of greater than or equal to 100 N and a metal coverage of at least 30%.

5. The stent delivery system of claim 4, wherein the stent is formed by weaving at least two kinds of first wires and second wires with different diameters, and wherein each of the first wires has a diameter of 20 μm to 150 μm and each of the second wires has a diameter of 150 μm to 800 μm.

6. The stent delivery system of claim 4, wherein the stent has the radial support force of 100 N to 600 N and the metal coverage of 30% to 90% in the natural release state.

7. The stent delivery system of claim 4, wherein the stent has the radial support force of greater than or equal to 400 N, preferably the radial support force of 400 N to 1000 N, and the metal coverage of 80% to 100%, preferably the metal coverage of 80% to 95%, in a release and axial maximum compression state.

8. The stent delivery system of claim 5, wherein the second wires comprise at least one first thick wire and at least one second thick wire with different diameters, and wherein the at least one first thick wire has a diameter of 150 μm to 300 μm, and the at least one second thick wire has a diameter of 300 μm to 600 μm.

9. The stent delivery system of claim 5, wherein the first wires comprise at least one first thin wire and at least one second thin wire with different diameters, and wherein the at least one first thin wire has a diameter of 20 μm to 100 μm, and the at least one second thin wire has a diameter of 100 μm to 150 μm.

10. The stent delivery system of claim 5, wherein the stent is formed by weaving 48 to 202 wires, wherein 4 or more wires, preferably 4 to 32 wires, of the 48 to 202 wires form the second wires, and remaining wires of the 48 to 202 wires form the first wires.

11. The stent delivery system of claim 8, wherein the stent is formed by weaving 48 to 202 wires, wherein 6 to 24 first thick wires and 4 to 24 second thick wires of the 48 to 202 wires form the second wires, and remaining wires of the 48 to 202 wires form the first wires, provided that a sum of a number of the first thick wires and a number of the second thick wires is less than or equal to 32.

12. The stent delivery system of claim 9, wherein the stent is formed by weaving 48 to 202 wires, wherein 4 to 32 wires of the 48 to 202 wires form the second wires, and remaining wires of the 48 to 202 wires form the first wires, and wherein the first wires comprise 32 to 166 first thin wires and 32 to 166 second thin wires, provided that a sum of a number of the first thin wires and a number of the second thin wires is less than or equal to 198.

13. The stent delivery system of claim 4, wherein the stent is further provided with at least one sparse mesh area formed only of the second wires, and the at least one sparse mesh area is arranged at a site of a corresponding treatment site having a branch artery after the stent is released.

14. The stent delivery system of claim 4, wherein the stent is used in an aorta comprising an abdominal aorta site, and the stent is internally provided with two common iliac artery stent fixing parts configured to fix left and right common iliac artery stents.

15. The stent delivery system of claim 14, wherein the common iliac artery stent fixing parts are arranged inside the stent and correspond to the abdominal aorta close to a bifurcation of left and right common iliac arteries, and wherein the two common iliac artery stent fixing parts are configured as two annuluses tangent to each other and are integrally formed with an inner wall of the stent.

16. The stent delivery system of claim 4, wherein the stent has a same diameter, or the stent has a variable diameter, wherein the stent has the diameter ranging from 20 mm to 60 mm.

17. The stent delivery system of claim 4, wherein the stent is provided with at least two layers of woven meshes.

18. The stent delivery system of claim 17, wherein an end of the stent is formed in a return weaving manner.
